(19) United States
(12) Patent Application Publication
(10) Pub. No.: US 2012/0225038 A1
(43) Pub. Date: Sep. 6, 2012

(54) MYELOID-DERIVED SUPPRESSOR CELLS GENERATED IN VITRO

(75) Inventors: Vincenzo Bronte, Abano Terme (Padova) (IT); Susanna Mandruzzato, AbanoTerme (Padova) (IT)

(73) Assignee: Istituto Oncologico Veneto, Padova (IT)

(21) Appl. No.: 13/146,823

(22) PCT Filed: Jan. 28, 2009
(86) PCT No.: PCT/IB2009/000137

§ 371 Date: Sep. 27, 2011

(51) Int. Cl. A61K 35/12; C12N 5/071

(52) U.S. Cl. 424/93.7; 435/377; 435/354; 435/366

(57) ABSTRACT

A population of myeloid-derived suppressor cells and the culture procedure to obtain these in vitro starting with bone marrow cells of mice, other animals and human beings, in the presence of specific cytokine combinations used to determine concentrations, is provided.

### TECHNICAL FIELD OF THE INVENTION

The present invention relates to a population of suppressor cells of myeloid origin and the procedure for obtaining it in vitro starting with mice marrow cells or human marrow aspiration.

### BACKGROUND ART

The immune system is composed of cells and molecules responsible for protecting a body from illnesses in general. Their response, coordinated with the introduction of foreign substances in the body, is the so-called immune response.

Nevertheless, the mechanisms which normally protect an individual from infections and from foreign substances at the same time can, in some circumstances, cause damage to the tissues in which the immune response occurs, causing a consequent illness. The best known pathologies of this type are: anaphylactic shock, allergies, self-immune illnesses and other pathological situations involving an exalted immune response.

The immune system has therefore evolved, developing a series of mechanisms able to prevent the damage caused by excessive or prolonged inflammation in the host. Some of these mechanisms which defend the host from the damage caused by the immune system itself comprise the generation and/or the expansion of cellular populations which, for example, negatively regulate the functions of one of the central components of the cell-mediate immune responses: the T lymphocytes.

One of these populations with negative regulatory function, recently called “suppressor cells of myeloid derivation”, is able to successfully inhibit the expansion of the T lymphocytes, both CD4⁺ and CD8⁺, and induces major dysfunctions of the immune system, both in the tumorous pathology context and in the course of acute and chronic infections.

The term “designation cluster” or “differentiation cluster”, abbreviated in CD, identifies a protocol used to identify molecules present on the cellular surface of different types of cells.

The name CD is used to classify such surface molecules, to each of which is attributed a number, and to identify cell markers that enable the cell to be classified according to the presence of such molecules on its surface.

The myeloid-derived suppressor cells are immature myeloid cells of hematopoietic derivation and can be traced in the blood, in the bone marrow, in the spleen and in the lymph nodes, but also in the tumorous micro-environment and in a context of strong immune activation, where they are able to suppress the immunity through complex paths of molecular activation that call for an increase of the metabolism of the amino acid L-arginine.

In mice, the characteristic markers on the surface of the myeloid-derived suppressor cells are Gr-I (an epitope common to the proteins Ly6G and Ly6C) and CD11b. In human beings on the other hand the myeloid line is mainly distinguished by the markers CD14 and CD15, though a general consensus has not yet been achieved regarding their fine characterization.

During the maturing process, the myeloid-derived suppressor cells can differentiate in a number of different cell types, such as, for example, macrophages, neutrophilic, monocytes, dendritic cells, etc.

The main functions performed by this cell population concern the suppression of the immune response mediated by the T lymphocytes. Consequent to the action of the suppressor cells, the incapacity of the effector T cells occurs to respond to the antigene, along with the increase in regulatory T cells and the production of growth factors, cytokines and other substances that regulate the growth and expansion cycle of other cells responsible for immune activity. The suppression of the responses of the T lymphocytes is also associated with tumorous growth and the myeloid-derived suppressor cells are involved in this process (Sica A. and Bronte V. J. Clin. Invest., 117:1155-66, 2007; Marigo, I., et al., Immunol Rev., 222:162-79, 2008).

For this reason and for their importance, the study of the myeloid-derived suppressor cells is undergoing strong expansion along with research, in human beings, for markers that can establish their definitive characterization.

Furthermore, the scientific community has recognized the clinical importance of the myeloid-derived suppressor cells and is therefore starting to invest in the cross research associated with the possible use of these cells.

Various pathologies do in fact exist that could take advantage of the use of these cells and consequently the development of a procedure able to obtain myeloid-derived suppressor cells in a systematic way could be advantageously exploited for patients with autoimmune complaints or patients undergoing heterologous organ transplant.

### SUMMARY OF THE INVENTION

One object of the invention is to upgrade the state of the previous art. Another object of the invention is to characterize in an in-depth way a population of myeloid-derived suppressor cells.

Another object of the invention is to characterize a population of myeloid-derived suppressor cells generated in vitro.

A further object of the present invention is to obtain a procedure for obtaining myeloid-derived suppressor cells in vitro.

Another object of the present invention is to obtain myeloid-derived suppressor cells starting with mouse marrow cells or human marrow aspiration.

A further object of the present invention is to use the myeloid-derived suppressor cells as immunosuppressive agents when the need exists to limit excessive immune response.

A further object of the present invention is to reduce the immune response mediated by the T lymphocytes.

A further object of the present invention is to use the myeloid-derived suppressor cells in case of autoimmune illnesses such as, for example but not limited to, type I diabetes (in the event of the pancreatic damage not being irremediable), rheumatoid arthritis, lupus erythematosus, vasculitis, autoimmune thyroiditis, multiple sclerosis or transplant rejection.

A further object of the present invention is the development of an effective immunotherapeutic approach to combat the above illnesses by administering myeloid-derived suppressor cells to patients suffering from such illnesses.

Another object is to use the present invention as a model to study the differentiation of myeloid-derived suppressor cells in vivo, in the cases of tumors or generalized infections.

A further object of the present invention is the development of an effective immunotherapeutic approach to combat the above illnesses through the inhibition of the differentiation and of the immunosuppressive activity of the myeloid-derived suppressor cells.

A further advantage of the present invention is the possibility of using the myeloid-derived suppressor cells as a useful instrument for evaluating new compounds that inhibit the suppressor action.

According to an aspect of the present invention, the characterization is envisaged of a population of myeloid-derived suppressor cells, of a procedure for obtaining them, and of a procedure for using them.

The dependent claims refer to preferred and advantageous forms of the invention.

### BRIEF DESCRIPTION OF THE DRAWINGS

Further characteristics and advantages of the invention will be more evident from the detailed description of a cell population of myeloid-derived suppressor cells, illustrated by way of example and not limited to, the attached drawings in which:

FIG. 1 represents the phenotype profile (obtained by means of cytofluorimetric analysis) of the murine myeloid-derived suppressor cells obtained from mouse bone marrow cultures of the C57BL/6 strain normally treated for four days with the cytokine combination Mix1 (FIG. 1C) or Mix2 (FIG. 1D) compared to the myeloid-derived suppressor cells isolated from the spleen of tumor-bearing mice MCA203 (fibrosarcoma) (FIG. 1A, positive control) and to the cells of mouse bone marrow of the C57BL/6 strain not treated with cytokine (FIG. 1B, negative control). In the axis Y of the left panel (indicated by 1) is indicated the marker Gr-I, in the axis Y of the right panel (indicated by 2) is represented the marker lymphocyte antigen 6 complex, locus G Ly6G, in the axis X of the left panel are indicated the markers CD11b (3), CD62 Ligando (4), receptor alpha for the interleukin 4 (IL-4R alpha) (5) and F4/80 (6); in the axis x of the right panel is indicated the marker lymphocyte antigen 6 complex, locus C Ly6C;

FIG. 2 is a graph representing the suppressor activity of the murine myeloid-derived suppressor cells, according to the present invention, in mixed lymphocyte cultures activated by means of peptide stimulation (MLPC, represented in A) or by means of halogenic stimulation (MLR, represented in B). The cytotoxic activity of the activated lymphocytes was assayed by means of the ⁵¹Cr release test. The left panel shows the graphs corresponding to 12% of myeloid-derived suppressor cells, in the central panel 6% of myeloid-derived suppressor cells and in the right panel 3% of myeloid-derived suppressor cells. In each graph, in the axis X, is indicated the dilution of the effector cells while in axis Y is indicated the ⁵¹Cr release percentage. The lines interspersed with black circles refer to MLPC, those interspersed with white circles refer to the myeloid-derived suppressor cells cultivated with the Mix1 and those interspersed with triangles refer to the myeloid-derived suppressor cells cultivated with the Mix2;

FIG. 3 represents the phenotypic analysis of human bone marrow cells after cell culture, according to the present invention.

In particular in (A) is represented the untreated marrow and in (B) the marrow treated with cytokine. In the graphs of the left panel, in the axis X is indicated the marker CD16 (8) and in the axis Y the marker CD11b (3), in the central panel of the axis X is indicated the marker CD15 (9) and in the axis Y the marker CD14 (10), in the right panel in the axis X is indicated the marker CD15 (9) and in the axis Y the marker IL-4R alpha (11);

FIG. 4 is the representation of the suppression of the proliferation of the PBMC responder marked Carboxy Fluorescein Succinimidyl Ester (CFSE) and stimulated with OKT3 and anti-CD28 in the presence of cells taken from marrow, according to the present invention. In particular in (A) are represented the data relating to the culture of the PBMC responder stimulated with OKT3 and anti-CD28; in (B) the PBMC responder stimulated by OKT3 and anti-CD28, with the addition of marrow cells cultivated in vitro without the addition of cytokine in a ratio of 1:1; in (C) the PBMC responder stimulated by OKT3 and anti-CD28, with the addition of marrow cells cultivated in vitro with the addition of cytokine granulocyte colony-stimulating factor (G-CSF) and granulocyte macrophage colony-stimulating factor (GM-CSF) (Mix1). The gate was positioned on the cells CD3+/CFSE+. In each graph, in the axis X is indicated the fluorescence intensity of the Carboxy Fluorescein Succinimidyl Ester (CFSE) and in the axis Y the number of cells;

FIG. 5 represents the experimental transplant pattern of pancreatic islets from syngeneic or allogenic mice and the subsequent adoptive transfer of myeloid-derived suppressor cells, according to the present invention. The numbers present inside the figure refer to the days on which the various actions are performed and on day 0 the transplant is made;

FIG. 6 represents the effect of the treatment with myeloid-derived suppressor cells, according to the present invention, on survival after the allogenic transplant of pancreatic islets (indicated by the Kaplan-Meier curves). In the axis X is indicated the time expressed in days and in the axis Y the survival percentage. The lines interspersed with circles refer to the myeloid-derived suppressor cells cultivated with Mix1, those interspersed with squares to the myeloid-derived suppressor cells cultivated with the Mix2 and those interspersed with triangles to the control;

FIG. 7 represents the histological evaluation of the transplants of allogenic islets after therapy with myeloid-derived suppressor cells, according to the present invention.

In particular in 1-3 is represented the syngeneic transplant, in 4-6 the allogeneic transplant on untreated receptors and in 7-9 the allogeneic transplant on receptors treated with myeloid-derived suppressor cells.

### DETAILED DESCRIPTION OF PREFERRED EMBODIMENTS

The present invention relates to a population of myeloid-derived suppressor cells and to the procedure for obtaining them in vitro.

In particular, such cells can be obtained from bone marrow or from other organs and tissues containing hematopoietic totipotent cells starting with mice and other mammals, including human beings.

Such cells are cultivated in the presence of various cytokine combinations in concentrations and for times long enough for the differentiation of the myeloid-derived suppressor cells.

The culture conditions occur mainly in the presence of suitable selected growth factors present in determinate conditions inside the culture medium.

The great interest shown for this type of cells stems from the possibility of using them to treat various pathologies including autoimmune pathologies and allogeneic responses, such as transplant rejection.

One of the main characteristics of these cells in fact is their capacity to reduce a lymphocyte response of T lymphocytes following the administering of myeloid-derived suppressor cells, and in particular to reduce the immune response of the T cells with respect to the host itself.

According to the present invention, such myeloid-derived suppressor cells are, in fact, suitable for use in the treatment of autoimmune illnesses, alloimmune responses or other pathologies involving a T lymphocyte response. Some examples of such pathological conditions are: type I diabetes, multiple sclerosis, lupus erythematosus, rheumatoid arthritis, transplant rejection, etc.

The experiments included below by way of explanation but not limited to the present invention, concern in particular the use of bone marrow of mice and human beings, but the marrow or other tissues or organs of other animals can be used.

Furthermore, in agreement with the present invention, various conventional cellular biology, molecular biology, microbiology, recombinant DNA and immunology techniques can be used, together with other techniques commonly present in laboratory practice.

### Generation of Myeloid-Derived Suppressor Cells

The haematopoietic cells are isolated from bone marrow of animals or human beings and are stimulated to differentiate in myeloid-derived suppressor cells through culture with different combinations of cytokines in particular concentrations.

The cells can be isolated and cultivated using different techniques known to the experts of the sector.

### Phenotypic And Functional Verification of the Myeloid-Derived Suppressor Cells

To make sure the cells obtained in vitro correspond to myeloid-derived suppressor cells, a phenotypic test is performed (which allows ascertaining the degree of differentiation of the myeloid-derived suppressor cells) and a function in vitro test to show the actual suppressing capacity of the cells thus obtained.

The tests shown below, in an exemplary but not limitative way, of the present invention, are based on the knowledge and experience acquired during the numerous years of study on this type of cells and on their in vivo generation following the onset of tumor in a host.

It appears clear nevertheless that such tests are not the only ones that can be performed to evaluate the phenotypic and functional characteristics of a cell population and that the most common procedures of molecular biology, cellular biology and genetic and biomolecular analysis can be applied with similar results (by way of example, analysis of transcriptome by means of genetic chip, microRNA profile analysis, analysis of metabolites produced by means of nuclear magnetic resonance, Elisa immunoenzymatic assays, Western blot protein analysis, secondary mediator flow analysis, etc.).

Such tests all fall within the protection scope of the present invention.

The present invention goes on to describe a number of examples which are purely illustrative and not limitative of the present invention.

Changes and variations may be known to the experts in the sector as regards the culture and cellular analysis procedure and all fall within the protection scope of the present invention.

### EXAMPLES

### Example 1

In vitro culture of murine bone marrow.

The bone marrow is recovered using known methods such as, for example, by means of “flushing” (which consists in inserting a syringe needle of a gauge corresponding to the medullary canal followed by forced injection of the liquid medium contained in the syringe to dislocate all the contents of the medullary canal itself) different strains, such as BALB/c or C57BL/6, from the tibias of mice. The most commonly used needles are the type 23G but different types of needles can also be used.

The cellular suspension thus obtained, including hematopoietic stem cells, undergoes the lysis of the red blood cells using a hypotonic solution. The lysing solutions that can be used, being careful of the type of cells being treated, are known and commonly used.

Some examples of the most commonly used lysing solutions are: the solution consisting of NH₄Cl 15.4 mM, KHCO₃ 0.1 mM, 0.01 mM EDTA, the ACK Lysing Solution (BioWhittaker, Walkersville, Md., USA), etc.

The cellular suspension from which the red blood cells have been eliminated is then resuspended in a suitable culture medium, preferably RPMI 1640 to which has been added 10% of fetal bovine serum (BioWhittaker), 2 mM of L-glutamine, 1 mM of sodium pyruvate, 1000 U/ml of streptomycin, 100 U/ml of penicillin and 20 μM of 2-β-mercaptoethanol,

Less additivated media can also be used; nevertheless, the cellular culture will be affected by this in terms of growth and survival.

To the cellular suspension so obtained are added the cytokines needed for cell differentiation and growth. The cytokines used are of two different mixes: the Mix1 containing 20-100 ng/ml (preferably 40 ng/ml) of recombinant murine granulocyte macrophage colony-stimulating factor (rmGM-CSF) and 20-100 ng/ml (preferably 40 ng/ml) of recombinant murine granulocyte colony-stimulating factor (rmG-CSF) and the Mix2 containing 20-100 ng/ml (preferably 40 ng/ml) of recombinant murine granulocyte macrophage colony-stimulating factor (rmGM-CSF) and 20-100 ng/ml (preferably 40 ng/ml) of recombinant murine interleukin 6 rmIL-6).

The cells are cultivated at the concentration of 1-4 million per plate with a preferred concentration of 2.5 million per petri plate (100 mm²) in 10 ml of above-described medium additivated with the mixes of cytokines at the above-mentioned concentrations, and incubated for 4 days at 37° C. and 5% CO₂.

Such culture concentrations, media and conditions merely represent a non-limitative example of the present invention.

Such culture procedure, thanks to the cytokines used in the above-described concentrations, permits the development and the differentiation of myeloid-derived suppressor cells according to the present invention.

Variations of these protocols which comprise different concentrations of cytokines or use of different plastics for in vitro culture are equally acceptable and concern the present invention.

### Example 2

In vitro culture of cells from human marrow aspiration.

The marrow blood used is a cytoaspiration with cytologic characteristics within normal limits. The marrow blood contains numerous erythroblasts and therefore undergoes the lyses of the red blood cells using a lysing solution.

Some examples of the most used lysing solutions are: the hypotonic solution consisting of NH₄Cl 15.4 mM, KHCO₃ 0.1 mM, 0.01 mM EDTA, the BD FACS™ Lysing Solution (BD), etc.

The cellular suspension from which the red blood cells have been eliminated is then re-suspended in a suitable culture medium, e.g., IMDM additivated with 15% FBS (Fetal Bovine Serum) or human serum, hepes buffer 0.01M (in 0.85% NaCl), penicillin 200 U/ml and streptomycin 200 U/ml.

Less additivated media can also be used, however the cellular culture is affected in terms of growth and survival.

The cells are then plated at the concentration of 0.5-1 million/ml with a preferred concentration of 0.75 million/ml in 24-well culture plates, in an end volume of 2 ml.

Such culture procedure, thanks to the cytokines used in the above-described concentrations, permits the development and the differentiation of myeloid-derived suppressor cells according to the present invention.

To the cellular suspension thus obtained are added the cytokines required for cellular differentiation and growth. The cytokines used are: human recombinant granulocyte colony-stimulating factor (rhG-CSF) at the concentration of 20-100 ng/ml (preferably 40 ng/ml) associated with human recombinant granulocyte macrophage colony-stimulating factor (rhGM-CSF) at the concentration of 20-100 ng/ml (preferably 40 ng/ml), or granulocyte macrophage colony-stimulating factor (rhGM-CSF) 20-100 ng/ml (preferably 40 ng/ml) associated with interleukin 6 (IL-6) 20-100 ng/ml (preferably 40 ng/ml).

The cells are cultivated at 37° C., 8% CO₂ for a period of time varying between 3 and 5 days, even though preferably the phenotypic and functional assays are made on the 4th culture day.

### Example 3

Phenotypic analysis by means of flow cytofluorimetry of the murine myeloid-derived suppressor cells.

The murine myeloid-derived suppressor cells obtained from the cultures are evaluated for the expression profile of a number of surface markers which is compared to the expression profile of the myeloid-derived suppressor cells obtained from the spleen of tumour-bearing animals (used as positive control) and fresh untreated marrow (used as negative control).

To prevent the non-specific binding of the antibodies, the most common laboratory procedures are applied. In particular, the cells can be pre-incubated for about 10 minutes at room temperature with the antibody 24G2 (ATCC, clone HB-197) mouse anti-receptor Fc-γ, that recognizes the extracellular domain of Fc-γRIII and murine RII.

Subsequently, the marking of the appropriate antibodies is performed.

The markers used in mice are (prevalently but not only): Gr-1, CD11c, CD62 Ligand, alpha receptor for interleukin 4 (IL4R alpha), F4/80, lymphocyte antigen 6 complex, locus C Ly6C, lymphocyte antigen 6 complex, locus G Ly6G, CD115, CD68, Arginase 1.

### Example 4

Phenotypic analysis by means of flow cytofluorimetry of the human myeloid-derived suppressor cells.

The human myeloid-derived suppressor cells obtained from the cultures are evaluated for the expression profile of a number of surface markers.

To saturate the non-specific attack sites of the antibodies the most common laboratory procedures are used. An example of such methods could be incubating with human poly IgG (0.2 mg/ml) depending on the indications of the producer.

The markers used in human beings to analyze the populations of immature myeloid-derived suppressor cells are: CD-14; CD11b; CD15; CD16; CD124 (IL4R alpha); CD115, Arginase 1; CD33; CD34, and the correct isotypic controls.

Marker and fluorochrome coupling is due solely to the fact of managing to perform multiple and contemporaneous markings inside the same sample and does not represent a limitative character of the present invention.

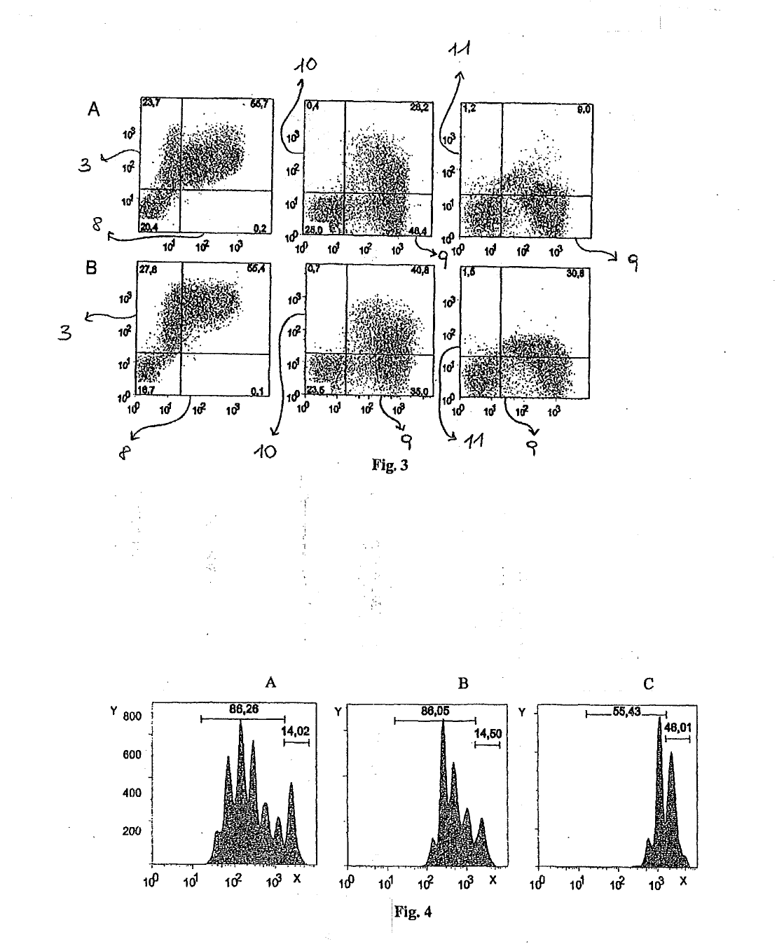

Such marking has made it possible to identify the populations present in the marrow samples, before and after treatment with cytokines, and to so assess which were the maturative profiles induced by the different cytokines and whether there was any expansion of an immature myeloid population expressing IL4-R alpha (FIG. 3).

### Example 5

Analysis of the suppressor activity of the myeloid-derived suppressor cells generated from cultures of lymphocyte murine bone marrow stimulated by peptides (MLPC) or by alloantigens.

The suppressor capacity on the lymphocytes T CD8+ of the murine myeloid-derived suppressor cells derived from the above-described bone marrow cultures can be evaluated in vitro by adding these cells as third part to mixed leukocytic cultures stimulated by peptide (MLPC).

In these MLPC the lymphocytes T CD8+ specific for the antigen gp100 from p-mel transgenic mice with specific TCR for gp100, were stimulated in vitro for 5 days and then tested as effector cells in a typical ⁵¹Cr release assay.

The suppressor capacity was also evaluated with regard to lymphocytes T CD8+ specific for other antigens such as HA (using transgenic mice C14) and for the antigen

OVA (using transgenic mice OT-1).

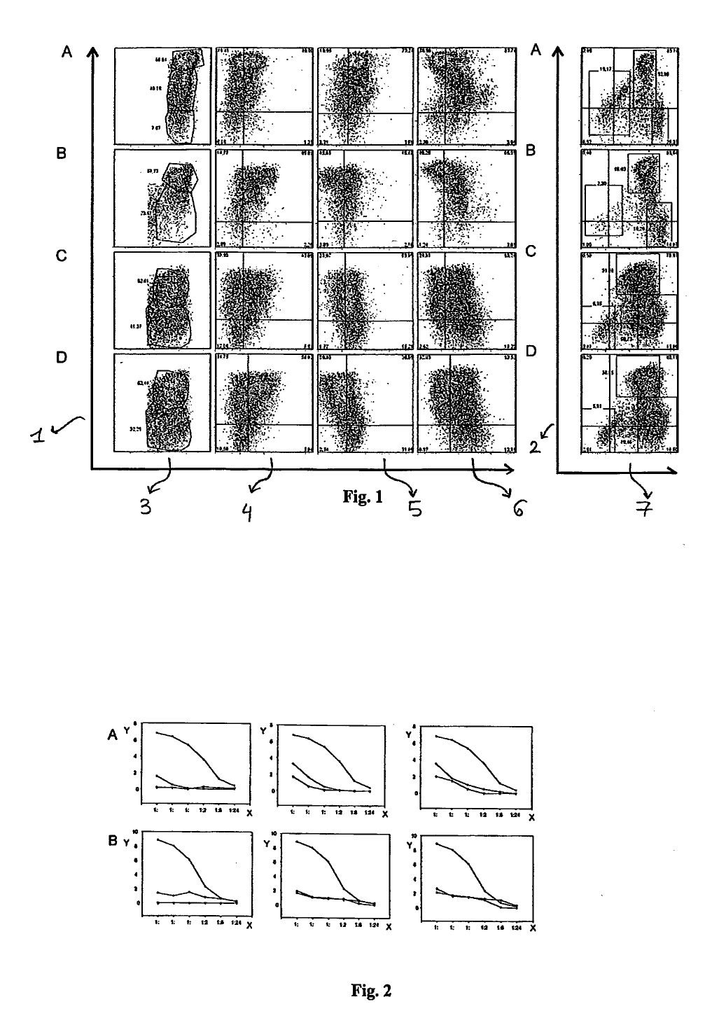

The myeloid-derived suppressor cells were added as third part to the MLPC in different concentrations: 12%, 6% and 3% (FIG. 2A).

Furthermore, the suppressor capacity of the myeloid-derived suppressor cells derived from bone marrow cultures on the lymphocytes T CD8+ was evaluated in vitro by adding these cells as third part to mixed leukocytic cultures (MLR), where splenocytes from C57BL/6 were stimulated in vitro for 5 days by gamma-irradiated allogeneic splenocytes from mice BALB/C and then tested as effector cells in ⁵¹Cr release assay.

The myeloid-derived suppressor cells were added as third part to the MLR in different concentrations: 12%, 6% and 3% (FIG. 2B).

### Example 6

Analysis of the suppressor activity of the myeloid-derived suppressor cells in lymphocyte cultures stimulated by mitogens or by alloantigens.

To evaluate the suppressor activity of the cells of marrow aspiration cultivated in vitro with the above-mentioned cytokines, two lymphocyte proliferation assays were made.

In the first assay, cultures were set up with mononuclear cells taken from peripheral blood (Peripheral Blood Mononuclear Cells, PBMC), allogeneic and stimulated with two monoclonal antibodies: 1) OKT3, a monoclonal antibody that recognizes the epsilon chain of the receptor complex of the CD3 whose function it is to stimulate the activation and the proliferation of the lymphocytes T in an independent way from the antigen; 2) a monoclonal antibody anti-CD28 directed against the co-stimulating molecule CD28. These antibodies allow the perfect stimulation of the lymphocytes and are both necessary to induce a good suppression on the part of the myeloid-derived suppressor cells.

Such cultures, in fact, occur in the presence or not of the cells derived from the marrow.

The proliferation of the PBMC is determined by means of marking with Carboxy Fluorescein Succinimidyl Ester.

By means of the cytofluorimetric analysis of the fluorescence of the PBMC and the consequent analysis of the data using specific software (ModFiT, Verity House), the number of cellular divisions can be monitored.

The PBMC responders to be marked with Carboxy Fluorescein Succinimidyl Ester are resuspended at the concentration of 20⁷/ml in PBS (Phosphate Buffered Saline) and mixed with a solution of Carboxy Fluorescein Succinimidyl Ester in PBS at the variable final concentration between 4 and 7 μM.

Such cells are then re-suspended at the desired concentration and plated in culture in 96 flat-bottom well plates previously covered with 1 μg/ml of OKT3 to which is added the antibody anti-CD28 at the concentration of 1 μg/well (1 mg/ml), in a final volume of 200 μl/well in complete culture medium.

The PBMC responders marked with Carboxy Fluorescein Succinimidyl Ester are then cultivated on the wells recovered with OKT3 and in the presence of anti-CD28 in the presence or not of the marrow cells treated with the cytokines, at the concentration of 10⁵ per population in each well (in a ratio of 1:1). The cellular culture is incubated for 4 days in an incubator at 37° C., with a concentration of CO₂ of 5%. The lymphocyte proliferation is evaluated on the third or fourth day of the culture by means of cytofluorimetry, evaluating the incorporation and the reduction in intensity of the Carboxy Fluorescein Succinimidyl Ester.

A second assay made to highlight the suppression mediated by myeloid-derived suppressor cells uses mixed lymphocyte cultures (MLR). In this way, the proliferation activity is evaluated of PBMC undergoing an allogeneic stimulation.

The PBMC responders are stimulated by the gamma-irradiated PBMC of another donor (called PBMC stimulator), which therefore have alloantigens able to trigger the lymphocyte response of the responders. In all the experiments carried out, a same pool of γ-irradiated PBMC was used, taken from three healthy donors.

The cells derived from marrow in the above-described conditions are gamma-irradiated and added as third part so as to determine their possible interference in the generation of an allogeneic response by the PBMC responders. The third part is added in dilution starting with a proportion 1:1 (responder: stimulator) up to a proportion 1:1/32. At the sixth day of culture the cells are marked with tritiated thymidine (³HTdR) and after 20-24 hours of incubation, the proliferation of the PBMC responders is quantified.

These two types of experiments allow us to analyze the effects of the myeloid-derived suppressor cells both in an activation model dependent on the alloantigen, and in an independent antigen activation model. Both the assays evidence the suppression, even though the assay with the mixed lymphocyte cultures shows a greater suppression compared to the activation with mitogens.

### Example 7

Myeloid-derived suppressor cells generated in vitro to limit the immune response against an allogeneic transplant.

The tolerogenic ability in vivo of the myeloid-derived suppressor cells derived from bone marrow was then evaluated by examining their therapeutic effect in mice BALB/c made diabetic and transplanted with pancreatic islets from animals of strain BALB/c (syngeneic) or C57BL/6 (allogeneic).

The glycaemia was measured three times a week and the animals were sacrificed when this parameter exceeded 250 mg/dL for at least three consecutive measurements.

The difference between the control mice that do not receive myeloid-derived suppressor cells and those receiving syngeneic myeloid-derived suppressor cells generated with the Mix2 is statistically significant (P<0.001).

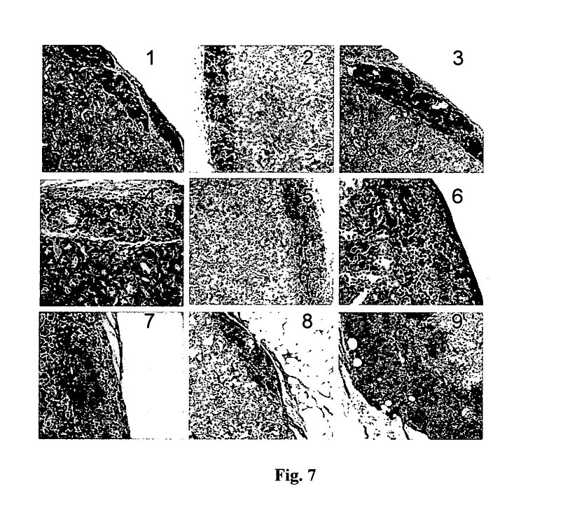

Upon rejection, or at determinate days after the transplant, the kidneys containing allogeneic islets were histologically examined to determine the state of the transplant at the time of the explant and the insulin content in the transplant itself (FIG. 7). These experiments can also be performed with other strains of animals that are allogeneic with one another, such as, for example, donors BALB/c and receptors 57BL/6.

As indicated above, such possibilities fall within the protection scope of the present invention.

### RESULTS AND DISCUSSION

FIG. 1 shows the phenotypic profile of the murine myeloid-derived suppressor cells obtained from cultures of bone marrow of mice C57BL/6 normally treated for four days with the cytokine combination Mix1 (FIG. 1C) or Mix2 (FIG. 1D) compared to the myeloid-derived suppressor cells isolated from the spleen of MCA203 tumor-bearing mice (FIG. 1A, positive control) and to the bone marrow cells of C57BL/6 mice normally not treated with the cytokines (FIG. 1B, negative control).

Analyzing the phenotypic profiles obtained and comparing them with the characteristic profile of the myeloid-derived suppressor cells, the cytokine concentrations to be used were established as well as the culture times needed for the culture to obtain the greatest expansion of the myeloid-derived suppressor cells, phenotypically similar to the myeloid-derived suppressor cells of tumor-bearing mice.

As appears evident from the example shown in FIG. 1C-D, by means of the cytokine treatments we have managed to obtain a phenotype identical to that of the myeloid-derived suppressor cells obtained from the tumour, shown in FIG. 1A. In particular, the expression of the examined markers shows a profile identical to the percentage of expression of the myeloid-derived suppressor cells obtained from mice with tumor and above all identical is the distribution of the various markers with respect to the GR-1 marker. Compared to the untreated marrow (FIG. 1B) the Gr-1^{low} population appears more expanded (low intensity of the Gr-1).

The expression of IL-4R alpha is more evident in the Gr-1^{low} fraction of the treated marrow compared to the untreated marrow. The expression of this marker is not only phenotypically but also functionally correlated to the suppressing ability of the myeloid-derived suppressor cells, as previously demonstrated (Gallina et al. J. Clin. Invest., 116:2777-2790, 2006).

Such data go to show that four days of treatment with the growth factors are perfect for obtaining a profile of markers of this type, and that along with the increase of culture days, there is also an increase in the expression of markers that correspond with dendritic cells, while there is a decrease in the expression of the marker CD62L, important for homing to the lymph nodes of these cells. The suppressing ability evaluated in vitro of the myeloid-derived suppressor cells obtained from bone marrow in various types of assays shows that these cells could be used in immunosuppression therapy.

FIG. 2 shows the suppressing activity of the murine myeloid-derived suppressor cells added in gradually reduced percentages (12%, 6% and 3%) in mixed lymphocyte cultures activated by means of peptide stimulation (MLPC, as shown in FIG. 2A) or by means of allogeneic stimulation (MLR, as shown in FIG. 2B).

Such cytotoxic activity with respect to the activated lymphocytes was assayed by means of the ⁵¹Cr release test.

These two types of experiments allow analyzing the effects of the myeloid-derived suppressor cells both in an activation model dependent on the alloantigen, and in an activation model with tumor antigen.

It can be seen that in both the experiments (FIGS. 2A and B), with the greater concentration of effector cells we find the maximum release of chromium, relating therefore to the higher percentage of lysed target cells. The inhibition percentage of the lysis after adding suppressor cells decreases along with a decrease in the percentage of myeloid-derived suppressor cells present in the culture. The situation is comparable for the two different cytokine combinations Mix1 and Mix2.

In all these cases therefore, it can be seen that the myeloid-derived suppressor cells obtained from the cultures of both the cytokine mixes induce immunosuppression in vitro.

FIG. 3 shows the phenotypic analysis of cells of human bone marrow after cellular culture in the presence (FIG. 3B) or absence (FIG. 3A) of the cytokines indicated in the text of the description. The maturation profile of mielo-monocytic populations was obtained using the marker pairs CD11b and CD16, or CD14 and CD15.

In fact, the combined use of different markers of the human myeloid cells, such as CD14 with CD15 and CD11b with CD16 (FIG. 3), allows characterizing the maturation stage of the analyzed cells, evaluating both the expression and the intensity with which these are expressed on the cellular surface (Terstappen, L. W., M. Safford, and M. R. Loken. Leukemia 4:657-663, 1990). Marking with monoclonal antibodies (mAb) anti-CD14 and anti-CD15 divides the mielo-monocytic populations into four distinct areas: the immature cells such as CFU-GM (units forming granulo-monocytic colonies), CFU-GEMM (units forming mixed granulocytic, erythroides, mielomonocytic and megakaryocytic colonies), hematopoietic stem cells (HSC), and also all the cells not belonging to the myeloid line such as the lymphocytes, do not express the molecules CD14 and CD15. Maturation in a mielo-monocytic sense leads to an increase in the expression of such markers, until differentiation into mature granulocytes and monocytes. The monocytes express high-intensity CD14, while CD15 is not expressed or low intensity. On the contrary, the granulocytes show a phenotypic profile opposite to that of the monocytes for these markers: in fact they express high-intensity CD15 while CD14 is not expressed or low intensity.

As illustrated in the representative experiment shown in FIG. 3, the marrow cultures without the addition of cytokines show a low expression of IL4R alpha (9.0%), while the addition to the cultures of marrow of the combination of granulocyte colony-stimulating factor (G-CSF) and granulocyte macrophage colony-stimulating factor (GM-CSF) induces a significant expansion of myeloid cells that express IL-4R alpha (30.8%).

It is interesting to observe that the phenotype obtained shows characteristics similar to the myeloid-derived suppressor cells expanded in patients with neoplasia (Mandruzzato et al., manuscript sent for publication), thus indicating that the expansion of marrow cells in vitro with the growth factors granulocyte colony-stimulating factor (G-CSF) and granulocyte macrophage colony-stimulating factor (GM-CSF) induces the mobilization of immature myeloid cells with the same phenotype as those observed in patients with carcinoma of the colon and melanoma (Mandruzzato et al., manuscript sent for publication).

As regards the other two monoclonal antibodies on the other hand (CD11b and CD16) which are used to study the maturation of the myeloid cells, these allow identifying in a more detailed way the various stages of maturation of the myelocyte sub-populations.

In fact, the myeloblast, progenitor cell of the myelocyte line, does not express the markers CD11b and CD16. The differentiation of the myeloblasts in mature neutrophil granulocytes contemplates an increase of the intensity of expression of the marker CD16. This increase is in relation to the maturating stage, in fact, the granulocytes-neutrophils, which represent the terminal stage of the differentiation of these cells, express CD16 at higher intensity compared to the intermediate maturating stage cells (Terstappen et al., 1990). All the differentiating stages of the granulocyte commitment (from the promyelocytes to the granulocytes) are not characterized by changes in intensity of expression for the marker CD11b; nevertheless, it can be seen that the intensity of fluorescence of the CD11b of the granulocyte precursors is lower in the marrow cultures not treated with the cytokines compared to those additivated with the growth factors granulocyte colony-stimulating factor (G-CSF) and granulocyte macrophage colony-stimulating factor (GM-CSF).

In the second suppression evaluation assay on lymphocyte proliferation (FIG. 4) it appears evident how the addition of cells derived from bone marrow cultivated for 4 days with the combination of granulocyte colony-stimulating factor (G-CSF) and granulocyte macrophage colony-stimulating factor (GM-CSF) (FIG. 4C) induces a considerable reduction in the percentage of cells that enter the cycle and, conversely, increases the percentage of cells that do not proliferate (evident in the first peak of each box on the right). The addition on the other hand of untreated marrow (FIG. 4B) does not change the proportion between cells in cycle and quiescent cells, but does delay the progression of the proliferating cells that enter the cycle.

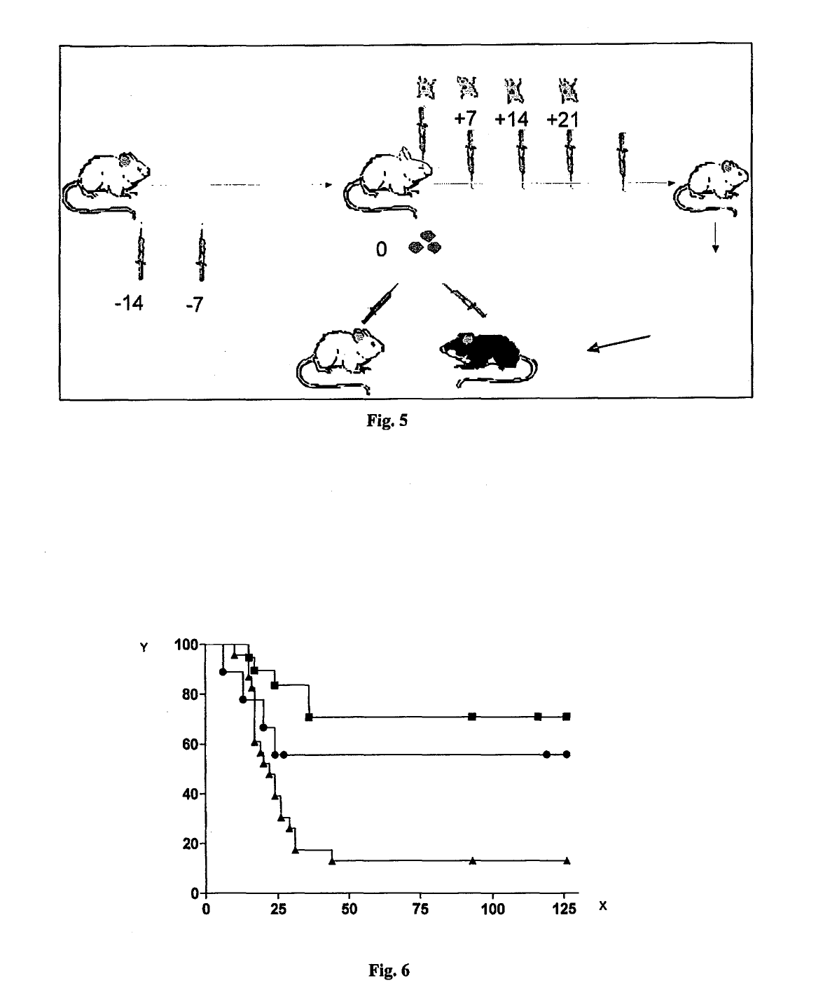

The evaluation of the immunosuppression capacity of the myeloid-derived suppressor cells was then verified in allogeneic transplants, the pattern of which is represented in FIG. 5. In particular, the mice BALB/c were made diabetic by means of two injections of streptozotocin (150 mg/kg). Once hyperglycemia had been induced in the mice, a subcapsular transplant of pancreatic islets was performed. The experimental groups included the transplant of pancreatic islets from syngeneic animals (BALB/c) or allogeneic animals (C57BL/6). The animals transplanted with allogeneic islets also received the endovenous adoptive transfer of 10×10⁶ syngeneic myeloid-derived suppressor cells derived from bone marrow. The adoptive transfer of myeloid-derived suppressor cells was performed once a week for 5 weeks starting on the fourth day after the transplant. The glycemia was measured three times a week and the animals were sacrificed when this exceeded 250 mg/dL. The administration schedule can vary.

As shown in FIG. 6, the survival of the transplanted mice is indicated by the Kaplan-Meier curves. The glycemia was measured three times a week and the animals were sacrificed when this parameter exceeded 250 mg/dL (for at least three consecutive measurements).

In this model, in the group of animals transplanted with allogeneic islets, the adoptive transfer of myeloid-derived suppressor cells derived from the marrow of animals BALB/c and obtained with the Mix1 and Mix2, (5 weekly injections starting on the same day as the transplant) increases long-term survival in a statistically significant way compared to the control group transplanted with allogeneic islets but in which the adoptive transfer does not occur. The syngeneic myeloid-derived suppressor cells generated with the Mix2 produce a highly significant statistical increase (P<0.001) compared to the control mice.

All the control animals transplanted with syngeneic islets (syngeneic transplant) remain normoglycemic during the period of observation.

The treatments with myeloid-derived suppressor cells to suppress the autoimmune response can be reasonably extended to other types of allogeneic transplant and other models, e.g., the model EAE of multiple sclerosis in mice, models of arthritis from autoimmunity towards collagen, models of autoimmune colitis, etc.

In FIG. 7, the histological evaluation can be displayed of the transplants of allogeneic islets after therapy with myeloid-derived suppressor cells.

In particular, after the syngeneic transplant (1-3) a strong coloration was found for the insulin in all the times evaluated after 166 days (represented in B). The receptors of untreated allogeneic transplant (4-6) have an average transplant survival of 17 days. Histologically, these transplants present an intense lymphocyte infiltrate and little or no coloration for the insulin. After treatment with the Mix-1 and 2 (7-9), the histological results vary between complete rejection (represented in 7), similar to untreated allogeneic transplant, and long-term survival after the transplant, with widespread coloration for the insulin, in the absence of lymphocyte infiltrate (8-9).

It can therefore be seen that the kidneys of animals treated with myeloid-derived suppressor cells derived from the bone marrow and which have survived allotransplant show widespread coloration for the insulin in the absence of any evident lymphocyte infiltrate.

1-103. (canceled)

104. Procedure for the culture and the differentiation of myeloid-derived suppressor cells comprising the following phases:
derivation of said myeloid-derived suppressor cells from bone marrow and/or other organs and tissues comprising hematopoietic totipotent stem cells from mouse and/or other mammals, including human beings,
obtaining, from said bone marrow, a cellular suspension comprising hematopoietic stem cells,
culture said cellular suspension in culture media additivated with the following cytokine mix:
granulocyte macrophage colony-stimulating factor and granulocyte colony-stimulating factor in concentrations and for times needed for the differentiation and the growth of said myeloid-derived suppressor cells,
differentiation of said myeloid-derived suppressor cells from said cellular suspension.

105. Procedure as claimed in claim 104, comprising a phase of elimination from said cellular suspension of the erythrocites contained in it by means of cellular lysis or other procedure.

106. Procedure as claimed in claim 104, in which said cytokine mix comprises each cytokine in a concentration varying between 20 and 100 ng/ml.

107. Procedure as claimed in claim 104, in which said cytokine mix comprises each cytokine in a concentration of 40 ng/ml.

108. Procedure as claimed in claim 104, wherein said culture phase of said cellular suspension obtained from said murine bone marrow occurs in vitro at a concentration of 0.1-0.4 million cells per ml of culture medium or of 0.25 million cells per ml of culture medium.

109. Procedure as claimed in claim 104, wherein said culture phase of said cellular suspension obtained from said human bone marrow occurs in vitro at a concentration of 0.5-1 million per ml of culture medium or of 0.75 million cells per ml of culture medium.

110. Procedure as claimed in claim 104, wherein said phase of culture of said cellular suspension lasts 3-7 days at 37° C. and 5% CO₂ or 4 days at 37° C. and 5% CO₂.

111. Myeloid-derived suppressor cells obtained with the procedure of claim 104.

112. Myeloid-derived suppressor cells according to claim 111, wherein said myeloid-derived suppressor cells are mouse cells or human being cells.

113. Myeloid-derived suppressor cells according to claim 111, in which said myeloid-derived suppressor cells show a phenotypic profile similar to the myeloid-derived suppressor cells isolated in vivo from tumor-bearing individuals.

114. Myeloid-derived suppressor cells according to claim 112, in which said murine myeloid-derived suppressor cells have on their surface the marker GR-1 expressed at low intensity.

115. Myeloid-derived suppressor cells according to claim 114, in which said cellular population having on its surface the marker GR-1 expressed at low intensity has on its surface a marker correlated with the suppressing ability of said myeloid-derived suppressor cells and/or in which said marker correlated with the suppressing ability of said myeloid-derived suppressor cells is the receptor alpha for the interleukin 4.

116. Myeloid-derived suppressor cells according to claim 112, in which said human myeloid-derived suppressor cells have on their surface the markers CD16−/CD11b+ and/or CD 15+/CD14+.

117. Myeloid-derived suppressor cells according to claim 116, in which said cellular population having on its surface the markers CD16−/CD11b+ and/or CD15+/CD14+ has on its surface a marker correlated with the suppressing ability of said myeloid-derived suppressor cells and/or said marker correlated with the suppressing ability of said myeloid-derived suppressor cells is the receptor alpha for the interleukin 4.

118. Myeloid-derived suppressor cells obtained with the procedure of claim 104, for the use as immunosuppressive agents for limiting the excessive immune response.

119. Myeloid-derived suppressor cells according to claim 118, in which said excessive immune response is mediated by the lymphocytes T or by other types of cells or molecules belonging to the immune system.

120. Myeloid-derived suppressor cells according to claim 118, in which said limitation of the immune response occurs by means of the suppression of the lymphocyte proliferation derived from peripheral blood mediated by said myeloid-derived suppressor cells or it occurs in mixed lymphocyte cultures additivated by means of a stimulation with an antigenic peptide or it occurs in mixed lymphocyte cultures additivated by means of a stimulation with mitogen agents and/or in an antigen-dependent way or it occurs in mixed lymphocyte cultures additivated by means of a stimulation with alloantigens.

121. Myeloid-derived suppressor cells according to claim 120, in which said antigenic peptide is recognised by the lymphocytes T CD8 or in which said mitogen agents comprise antibodies that recognise the chain E of the receptorial complex of the CD3 of the lymphocytes, e.g., OKT3, or which recognize a co-stimulating molecule, e.g., CD28, or other substances whose function is to trigger the development and the proliferation of the lymphocytes T, or in which said alloantigens are chosen among the antigens belonging to an individual but recognised as foreign by another individual of the same species, e.g., mononuclear cells of peripheral blood of a different individual compared to the receptor individual.

122. Myeloid-derived suppressor cells according to claim 118, in which said myeloid-derived suppressor cells are used as immunosuppressive agents for treating autoimmune disorders, such as rheumatoid arthritis, type I diabetes, multiple sclerosis, lupus erythematosus, rheumatoid arthritis and any other autoimmune disorder.

123. Myeloid-derived suppressor cells according to claim 118, in which said myeloid-derived suppressor cells are used as immunosuppressive agents for treating alloimmune responses, such as the rejection of transplants, host-versus-graft disease and any other type of alloimmune response.

124. Myeloid-derived suppressor cells according to claim 118, in which said myeloid-derived suppressor cells derive from mice and are syngeneic.

125. Myeloid-derived suppressor cells according to claim 124, in which said syngeneic myeloid-derived suppressor cells are transferred adoptively for the suppression of the lymphocyte proliferation in diabetic mice receiving a transplant of allogeneic pancreatic islets.

126. Myeloid-derived suppressor cells according to claim 125, in which said adoptive transfer of said syngeneic myeloid-derived suppressor cells prolongs the survival of the receptor mice with respect to the control group.

127. Procedure according to claim 125, in which said adoptive transfer of said myeloid-derived suppressor cells can be used as immunosuppressive agents in other types of allogeneic transplant.

128. Myeloid-derived suppressor cells according to claim 120, in which said lymphocyte suppression mediated by said myeloid-derived suppressor cells occurs in individuals receiving a transplant or presenting an autoimmune disorder or an excessive immune response and/or any study model and/or in vivo in cases of neoplasia and generalized infections.

129. Myeloid-derived suppressor cells according to claim 111, in which said myeloid-derived suppressor cells are present in the tumorous microenvironment or can be obtained in vivo by administering to patients granulocyte macrophage colony-stimulating factor associated with granulocyte colony-stimulating factor.

130. Myeloid-derived suppressor cells according to claim 111, in which said myeloid-derived suppressor cells can be obtained by engineering or other suitable method

131. Myeloid-derived suppressor cells obtained with the procedure of claim 104, for use as a means for evaluating the action of new compounds that inhibit the suppressor action or of any other compound the action of which interferes with the action of said myeloid-derived suppressor cells.

132. Procedure for the treatment of an autoimmune disorder, and/or of an alloimmune response, and/or of an excessive immune response in an individual, characterized by the administering of myeloid-derived suppressor cells obtained by the procedure of claim 104.

133. Procedure according to claim 132, in which said myeloid-derived suppressor cells are administered as immunosuppressive agents for limiting the excessive immune response and/or for obtaining lymphocyte suppression.

134. Procedure according to claim 132, in which said autoimmune disorders are rheumatoid arthritis, type I diabetes, multiple sclerosis, lupus erythematosus, rheumatoid arthritis and any other autoimmune illness or in which said alloimmune responses are transplant rejection, host-versus-graft disease and any other type of alloimmune response.

135. Procedure according to claim 133, in which said lymphocyte suppression mediated by said administering of said myeloid-derived suppressor cells occurs in individuals receiving a transplant and/or having an autoimmune disorder and/or an excessive immune response and/or any study model.

136. Use of myeloid-derived suppressor cells obtained by the procedure of claim 104 as a system suitable for highlighting the effectiveness and/or the effects of drugs and/or treatments with antineoplastic action.
